(19) United States
(12) Patent Application Publication
(10) Pub. No.: US 2025/0116655 A1
(43) Pub. Date: Apr. 10, 2025

(54) VIRUS DETECTION BY REFLECTED LIGHT

(71) Applicant: SARSIECO LLC, New York, NY (US)

(72) Inventors: Bo Gehring, Willow, NY (US); Jib Ray, Petrolia, CA (US)

(21) Appl. No.: 18/793,802

(22) Filed: Aug. 3, 2024

Related U.S. Application Data
(63) Continuation-in-part of application No. 17/587,334, filed on Jan. 28, 2022.
(60) Provisional application No. 63/199,875, filed on Jan. 29, 2021.

(51) Int. Cl. G01N 33/497; G01N 21/51

(52) U.S. Cl. CPC G01N 33/4975; G01N 21/51; G01N 2201/062; G01N 2333/005

(57) ABSTRACT

Described is a device and method for detecting enveloped viruses by using light reflected from enveloped viruses.

### CROSS-REFERENCE TO RELATED APPLICATIONS

This application is a continuation-in-part application of U.S. application Ser. No. 17/587,334, filed on Jan. 28, 2022, which claims priority benefit of U.S. Provisional Patent Application Ser. No. 63/199,875 filed on Jan. 29, 2021, and entitled “ULTRAFAST CORONAVIRUS DETECTOR.” The disclosures of the aforementioned Provisional Patent Application Ser. No. 63/199,875 and U.S. patent application Ser. No. 16/740,166 are hereby incorporated by reference in their entirety.

### BACKGROUND

Current methods of testing for viruses typically are either molecular tests that detect the virus's genetic material or antigen tests that detect specific proteins from the virus.

In today's most widely used test, the antigen protein test, a swab is swirled in the subject's nose. Nasal fluid absorbed by the swab is mixed with a reagent. The resulting mix is dripped onto a telltale strip which after a fifteen-minute wait time displays a variable color to tell whether the subject is infected with COVID-19 or not. The so-called “gold standard” RT-PCR molecular tests can take hours to display results.

### SUMMARY

The inventors have recognized that the significant shortcoming of these biochemical methods is that they cannot effectively process groups or crowds and what is needed are rapid, simple, screening tests. Observing that SARS-COV-2 superspreader events, and their consequent death toll, were caused by airborne microdroplets encompassing virus, and that many respiratory viruses, including SARS-COV-2, reproduce by extruding a lipid envelope which encases new virus, the inventors recognized that there would be an opportunity to develop a rapid test for such enveloped viruses because the lipid envelope reflects light like a soap bubble. The device and methods described herein, in various embodiments, illuminate a subject's breath and determine the degree of infection from the amount of light reflected from the virus microdroplets. The understanding that illuminating a subject's breath and determining the degree of infection from the amount of light reflected provides for methods, devices, and apparatus for rapid determination of enveloped viruses in subjects. This approach has valuable applications for detecting virus in humans and other animals. Farms and places where people gather will have a strong need for these rapid tests. This is especially the case as we are likely to face an increase in viral pandemics as our environment becomes destabilized due to climate change.

By contrast, the biochemical methods that are currently in use require minutes to hours waiting time per test. When individuals of unknown infection status wait together for their results, there is high risk of contagious transmission. The biochemical methods also generate contaminated disposables such as used swabs and reagents. The most significant shortcoming of the prior art, both antigen and PCR, is that it takes minutes to hours to test only one person. The testing of a group quickly becomes untenable with even the fastest biochemical methods. For example, consider one hundred passengers lined up to board an airplane where each passenger's pre-boarding coronavirus test takes five minutes. With the fastest biochemical device, at best it takes 5 minutes times 100 passengers equals 500 minutes or more than eight hours for all to board. Even with ten testing devices the wait is still about an hour.

Provided herein, in various embodiments, are methods, devices, and systems that can detect enveloped virus using reflected light from enveloped viruses. The various embodiments described herein solve the problem of how to rapidly test for enveloped virus. Rather than biochemical analysis, viruses are detected from reflected light from enveloped viruses based on optical physics. Results can be obtain in seconds, generally under ten seconds, and more than two hundred times as fast as biochemical tests. In various embodiments, results can be obtained in about 4 seconds. In some embodiments, results can be obtained in about 2 seconds. In some embodiments, results can be obtained in less than 2 seconds.

In some embodiments, provided is a device for detecting an enveloped virus in a subject, comprising a sample space through which breath from the subject passes, a light source positioned to shine through the sample space, and one or more sensors positioned to measure the light from the light source which is reflected from the virus in the sample space. In some embodiments, the sample space comprises a tube. The tube generally can be introduced into a bore in the device which forms a sample space. In some embodiments, the tube is a disposable tube. In some embodiments, the subject is a mammal. In some embodiments, the subject is a human. In some embodiments, the subject is a cow. In some embodiments, the subject is a bird. In some embodiments, the subject is selected from a cow, a sheep, a goat, a pig, or a chicken. In some embodiments, the one or more sensors are digital optical sensors. In some embodiments, the one or more sensors optical sensors have an accuracy of around one part per 65536 and 16 bit integrated circuits. In some embodiments, the light source a LED (light emitting diode). In some embodiments, the device further comprises a purging fan. In some embodiments the device further comprises a filter. In some embodiment, the filter is configured to capture virus. In some embodiments the filter can capture virus particles for further analysis. In some embodiments the device further comprises a molecular test to identify the enveloped virus detected. In some embodiments, a molecular test to identify enveloped viruses detected is separate from the device. In some embodiments, the molecular test is selected from reverse transcriptase polymerase chain reaction (RT-PCR), antigen detection, nucleic acid amplification tests, and serology tests, or combinations thereof. In some embodiments, the device further comprises wireless means to communicate externally. In some embodiments, the wireless means is a Bluetooth®. In some embodiments, the device comprises a smartphone.

In some embodiments, provided herein is a method of detecting an enveloped virus in a subject, comprising having the subject breath into a sample space; shining light on the sample space; and measuring the reflected light in the sample space to determine the presence of the enveloped virus. In some embodiments, the method further comprises quantifying the enveloped virus. In some embodiments, the method further comprises identifying the enveloped virus using a molecular test. In some embodiments, the molecular test is selected from reverse transcriptase polymerase chain reaction (RT-PCR), antigen detection, nucleic acid amplification tests, and serology tests, or combinations thereof. In some embodiment, the enveloped virus detected is selected from the group consisting of SARS-COV-2, Influenzas A and B, Severe Acute Respiratory Syndrome (SARS), Middle East Respiratory Syndrome (MERS), Respiratory Syncytial Virus (RSV), H5N1 Avian Influenza, variants thereof, and combinations thereof. In some embodiments, the light is in the range of 380 to 780 nm. In some embodiments, In some embodiments, the subject is a mammal. In some embodiments, the subject is a human. In some embodiments, the subject is a cow. In some embodiments, the subject is a bird. In some embodiments, the subject is selected from a cow, a sheep, a goat, a pig, or a chicken.

In some embodiments, provided herein is a device for testing water for enveloped viruses, comprising a sample space for presenting a water samples; a light source positioned to shine through the sample space, and one or more sensors positioned to measure the light from the light source which is reflected from the enveloped virus in the sample space. In some embodiments the samples space is a central bore adapted for receiving running water. In some embodiments, the sample space further comprises a transparent test tube for providing a water sample. In some embodiments the test tube is glass.

Envelope viruses that can be detected using the devices and methods provided herein include, for example, SARS-COV-2, Influenzas A and B, Severe Acute Respiratory Syndrome (SARS), Middle East Respiratory Syndrome (MERS), Respiratory Syncytial Virus (RSV), H5N1 Avian Influenza, variants thereof, and combinations thereof.

In operation, for example, a single device, in accordance with various embodiments described herein, can test airline passengers as quickly as they can file through a boarding gate. No waiting area is required. The inventors' disclosure can transform team sports, classrooms, hospital emergency rooms, religious services, workspaces, and many others where groups gather, with respect to handling detection of virus.

In various embodiments, enveloped viruses can be detected by reflected light using a device comprising a sample space for detection, a light source, and a sensor. The sample space can be a tube, for example, through which breath of an animal is passed or water is passed. Light in the range of infrared to ultraviolet, including the visible spectrum which is typically between 380 and 780 nanometers, is then passed through the space. The measured strength of reflection of light, as compared with a control, indicates the presence and amount of the virus. In various embodiments, a device called SARSIE is used. As used herein, “SARSIE” or “Sarsie” refers to a device for detecting enveloped viruses by reflected light as described herein in various embodiments. Sarsie, or SARSIE, is a name for the device described herein in various embodiments for detecting envelope viruses. “SARSIE”, “Sarsie”, and device for detecting envelope viruses using reflected light, and various other expressions of this concept, are used interchangeably throughout this application. SARSIE comprises a sample space for detection, a light source, and a sensor. The sample space can be a tube, for example, through which breath of an animal is passed or water is passed. Light in the range of infrared to ultraviolet, including the visible spectrum which is typically between 380 and 780 nanometers, is then passed through the space. The measured strength of reflection of light, as compared with a control, indicates the presence and amount of the virus. A variety of sensors known to persons of skill in the art can be used. Digital optical light sensors, such as those made by Analog Devices, can be used in various embodiments. In various embodiments, optical sensors having 16 bit integrated circuits and an accuracy of around one part per 65536 can be used. The sample space in devices, in accordance with various embodiments, can be a tube. A tubular bore is simple to manufacture and set up, however, the sample space can be many other shapes, such as rectangular with rounded corners to fit a specific hardware requirement. The bore design could include baffles or protuberances to focus a source emission, such as an LED. In various embodiments, the light source is a LED (light emitting diode) integrated circuit. LEDs can be tailored to specific frequencies.

In various embodiments, provided herein is a method for detecting enveloped viruses, comprising collecting the breath of an animal in a sample space, passing light through the sample space, and measuring the reflected light, wherein the strength of the reflection of light indicates the presence and amount of the virus.

In various embodiments, a device called SARSIE is used. As used herein, “SARSIE” or “Sarsie” refers to a device, in accordance with various embodiments described herein, for detecting enveloped viruses by reflected light as described herein in various embodiments. SARSIE comprises a sample space for detection, a light source, and a sensor. The sample space can be a tube, for example, through which breath of an animal is passed or water is passed. Light in the range of infrared to ultraviolet, including the visible spectrum which is typically between 380 and 780 nanometers, is then passed through the space. The measured strength of reflection of light, as compared with a control, indicates the presence and amount of the virus. A variety of sensors known to persons of skill in the art can be used with SARSIE, or other devices described herein. Digital optical light sensors, such as those made by Analog Devices, can be used in various embodiments. In various embodiments, optical sensors having 16 bit integrated circuits and an accuracy of around one part per 65536 can be used. The sample space in devices, in accordance with various embodiments, can be a tube. A tubular bore is simple to manufacture and set up, however, the sample space can be many other shapes, such as rectangular with rounded corners to fit a specific hardware requirement. The bore design could include baffles or protuberances to focus a source emission, such as an LED. In various embodiments, the light source is a LED (light emitting diode) integrated circuit. LEDs can be tailored to specific frequencies.

In some embodiments, SARSIE, or other device in accordance with embodiments described herein, is a battery-powered sealed device about the size of a computer mouse and weighing about six ounces. In some embodiments, there is a pushbutton and three LEDs indicating respectively TESTING (white), NEGATIVE (green), and POSITIVE (red).

In some embodiments, a human subject presses a button to start a test and then exhales once through the device's tube, or other sample space. In less than ten seconds the appropriate LED lights up to indicate whether the subject tested positive (red) or negative (green) for COVID-19. The result can also be transmitted, in various embodiments, via the device's built-in wi-fi, or Bluetooth®, to a linked smartphone or laptop application along with other data which may be useful such as the device's GPS geolocation for contact tracing. In some embodiments, a USB port is provided for charging batteries and programming.

In operation, in some embodiments, as shown in FIG. 1, FIG. 2., and FIG. 3, the device's LED light source 14 on PC Board 3 shines crosswise to the subject's breath flowing through the breath passage. Sensor photodiodes 12 and 13 on PC Board 3 measure the intensity of the light reflected as described above. Analysis to determine whether the subject is negative or positive is computed by the CPU on PC Board 3. The test result is displayed by an indicator LED 5 and saved to non-volatile memory on PC Board 3. Data and results can be sent, in some embodiments, immediately via wireless communication 8, to a linked smartphone app or cloud storage to assist in automated contact tracing.

In various embodiments, provided herein is a device which tests a subject's breath for the presence of an envelope virus such as coronavirus such as COVID-19, comprising: an enclosure with a passage through which the subject's breath passes, a light source positioned to shine through the subject's breath as the subject's breath passes through the passage, one or more sensors positioned so as to measure the amount of light produced by the light source which is reflected into the sensor(s) from the envelope virus particles in the subject's breath. In some embodiments, the device further comprises a disposable tube that is inserted in the passage and that can be discarded after each use. In some embodiments, the device comprises a purging fan that can be used to cleanse the passage of virus after each use. In some embodiments the device comprises a filter that can capture virus particles drawn into the purging fan. In some embodiments, the device comprises wireless means for communicating externally. In some embodiments, the device further comprises a molecular test that can be used to identify the virus.

It will be clear to those skilled in the art that embodiments such as device shape and construction, number and arrangement of sensors, light source LED details, purge fan details, direct radio link in addition to Bluetooth, and other such embodiments can be adjusted if needed for any of several applications within the scope

Because of the rapidity in which detection of virus can be achieved, a single one of of the devices described herein can test airline passengers as quickly as they can file through a boarding gate, like a metal detector, and immediately flag passengers who should not be allowed to board.

In a hospital setting, the methods and devices described herein, can be employed during the hospital triage processes and patients who test positive could be immediately cohorted into a separate waiting room, thus decreasing the chance of inadvertently infecting others.

By enabling safe gathering with less fear of infection, the invention in its various embodiments and implementations can transform social spaces such as dinner parties, sports events, religious services, classrooms, workspaces, and many other types of gatherings.

In various embodiments, described herein, are devices and methods for testing water for enveloped viruses by measuring reflected light from water samples.

### BRIEF DESCRIPTION OF THE DRAWINGS

The following detailed description, given by way of example and not intended to limit the invention solely to the embodiments described herein, will best be understood in conjunction with the accompanying drawings, in which:

FIG. 1 shows an exterior view. Visible are the outer enclosure 1, disposable tube 2, indicator LED 5, actuator button 6, USB cable 7, and purging fan assembly 9;

FIG. 2 removes the upper halves of the outer enclosure 1 and purging fan 9 to reveal the following features: PC board 3 which includes non-volatile memory and CPU, sensors 12 and 13, red LED 14, and also battery 4, Bluetooth antenna 8, breath filter 10, and purging fan motor 11;

FIG. 3 removes the disposable tube 2 and other components to clearly show the lower half of the breath passage;

FIG. 4 graphs five 50 millisecond breath tests of four subjects and one “Room Tone” which is the floor value for this test. The “DELTA” value shows a clear separation between positive and negative;

FIG. 5 shows that the sensors are accurate over time. Each reading is indicated with ‘x’;

FIG. 6 indicates a live subject positive with a dotted line and shows the trip point. The readings are over five days;

FIG. 7 shows the same data as FIG. 6 but inverted for easier interpretation;

FIG. 8 provides a comparison of negative patient BR with infected patient S. The positive reading is robust, that is stable, over five days;

FIG. 9 shows a close-in (magnified) data comparing plain versus 1000:1 surfactant. The average lines show the separation;

FIG. 10 provides readings named as shown, 694 readings showing open device versus Infected Patient S extrapolated demonstrating that the device can work “full speed” (button looked off) without any bad readings;

FIG. 11 shows six data captures comparing POSITIVE Patient S with Negative Individual B with the detector capped. Note that there is clear separation;

FIG. 12 provides detail of the operation of the breath sensor used in Sarsie and other embodiments described herein. LED RED is traced by wavelength relative intensity and wavelength by responsivity;

FIG. 13 compares HOURLY test readings to extrapolated Infected Patient S;

FIG. 14 shows an embodiment for testing water for enveloped viruses;

FIG. 15 shows an inside view of an embodiment for testing water for enveloped viruses;

FIG. 16 shows a smart phone embodiment of the device;

FIG. 17 shows a device that further comprises a molecular test; and

FIG. 18 is an illustration with a cow.

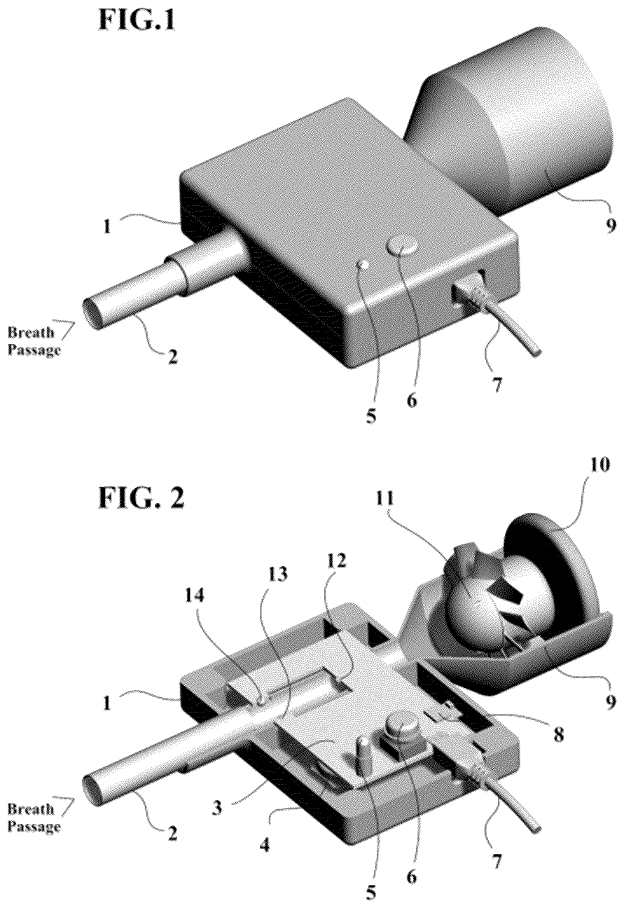

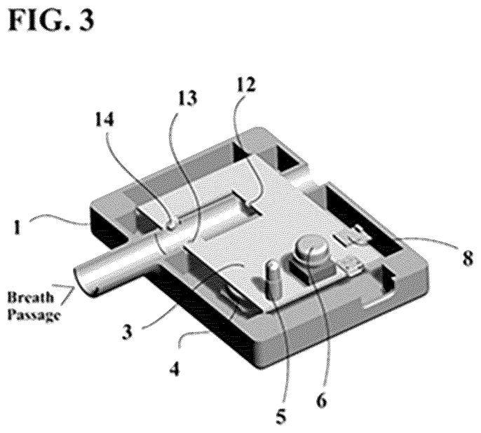

### DETAILED DESCRIPTION

Before turning to the figures, which illustrate the exemplary embodiments in detail, it should be understood that the present disclosure is not limited to the details or methodology set forth in the description or illustrated in the figures. It should also be understood that the terminology is for the purpose of description only and should not be regarded as limiting. An effort has been made to use the same or like reference numbers throughout the drawings to refer to the same or like parts.

As used herein, a “SARSIE” or “Sarsie” refers to a device for detecting enveloped viruses by reflected light as described herein in various embodiments. SARSIE comprises a sample space for detection, a light source, and a sensor. The sample space can, in some embodiments, be a bore enclosed within a housing. The sample space can be a tube, for example, through which breath of an animal is passed or water is passed. Light in the range of infrared to ultraviolet, including the visible spectrum which is typically between 380 and 780 nanometers, radiates through the space. The measured strength of reflection of light, as compared with a control, indicates the presence and amount of the virus.

A variety of sensors known to persons of skill in the art can be used with SARSIE, or other devices described herein. Digital optical light sensors, such as those made by Analog Devices Inc., can be used in various embodiments. In various embodiments, optical sensors having 16 bit light detecting integrated circuits and an accuracy of around one part per 65536 can be used. In various embodiments, these sensors have a sensing cone or aperture from about 20 degrees to 180 degrees. In some embodiments, a midrange value of about 70 degrees is used.

In various embodiments, Sarsie, or the device, is enclosed in a housing which can be 3D printed, injection molded, or made using other means of fabrication. The housing has a bore through which the subject's breath travels. A tubular bore is the easiest to manufacture and, in use, the easiest to clean or sterilize. The sample space in devices, in accordance with various embodiments, can be a tube. A tubular bore is simple to manufacture and set up, however, the sample space can be many other shapes, such as rectangular with rounded corners to fit a specific hardware design. The bore design could include baffles or protuberances to focus a source emission, such as an LED. Detection in water has the same features and actions.

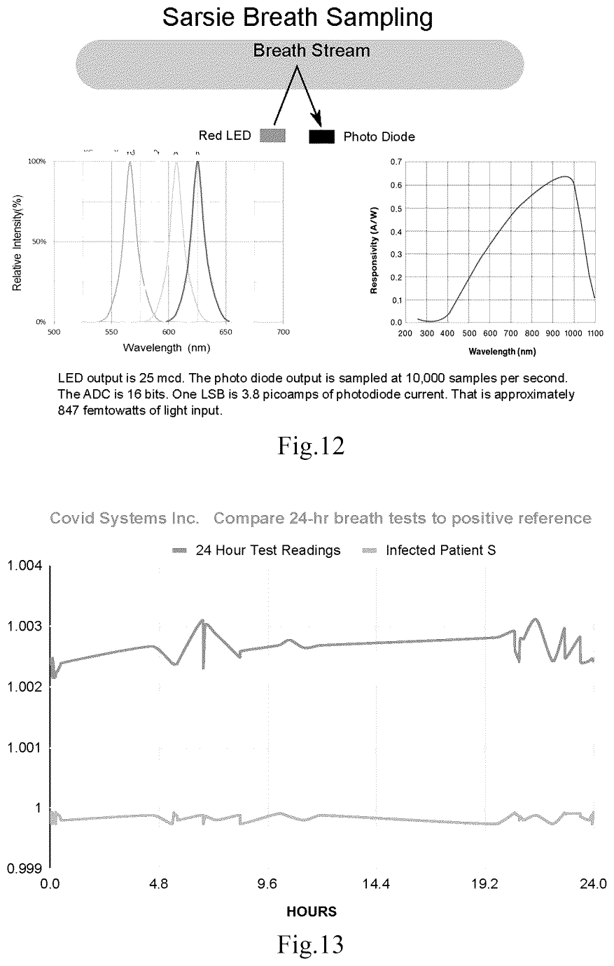

The light source is, in various embodiments, an LED (light emitting diode) integrated circuit. This LED can be tailored to specific frequencies, and, in various embodiments, be set to a specific frequency. Additional light sources can be added in some embodiments to enhance the accuracy of a reading by UV or infrared sensing. FIG. 12 shows results with a red LED light source, that can, in some embodiments, be used with a standalone device. The light source has a cone of radiation with values similar described to the sensor described herein in various embodiments. In some embodiments, the light source will subtend 70 degrees more or less. In some embodiments, the sensor and light source are positioned closely side by side on the device's circuit board. Side by side positioning has been shown to produce accurate reflection readings. The FIGS. show the amount of light reflected from airborne virus microdroplets as they pass through the invention's central bore. This value is the viral load at the moment of testing.

In some embodiments, for purposes of comparison, a positive infection can be simulated with an oral rinse of 1000:1 water to a surfactant. This appears as “surfactant” in the drawing descriptions. Live human subjects are identified with initials. FIG. 12 shows the detailed operation and breath sampling in Sarsie hardware.

In these graphs, the Y axis values always represent the same kind of value, viral load, but for reasons of clarity and comparison the presentation may be scaled or the value inverted. This is shown on each graph. Accuracy of the Sarsie sensors is at least 16 bits.

These drawings show the amount of light reflected from airborne virus microdroplets as they pass through the invention's central bore. This value is the viral load at the moment of testing. It is also possible, for purposes of comparison, in various embodiments, to simulate positive infection with an oral rinse of 1000:1 water to a surfactant. This appears as “surfactant” in the drawing descriptions. Live human subjects are identified with initials. FIG. 12 shows the detailed operation and breath sampling of the Sarsie hardware.

In these graphs, the Y axis values always represent the same kind of value, viral load, but for reasons of clarity and comparison the presentation may be scaled or the value inverted. This is shown on each graph. Accuracy of the Sarsie sensors is at least 16 bits.

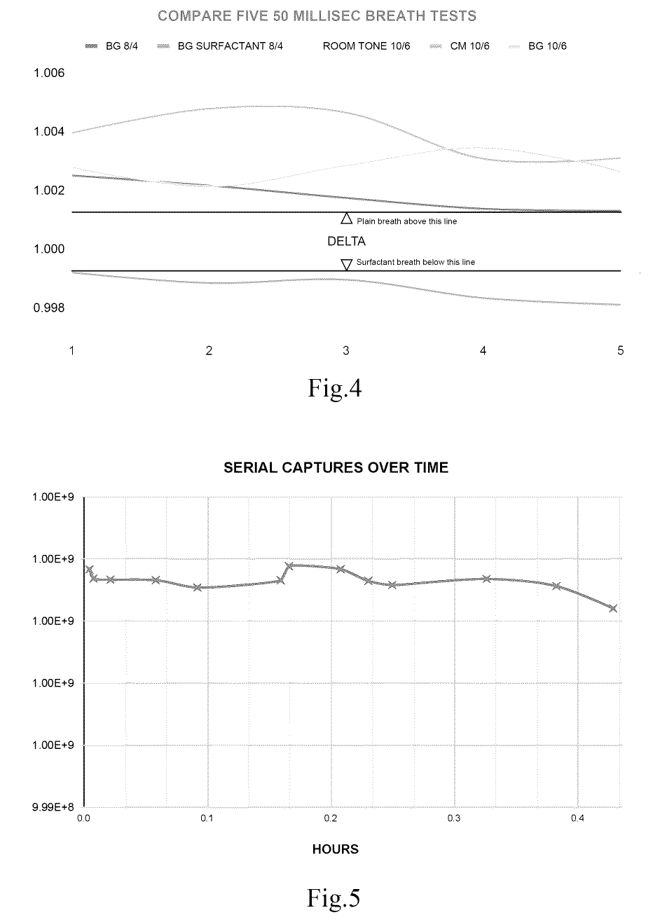

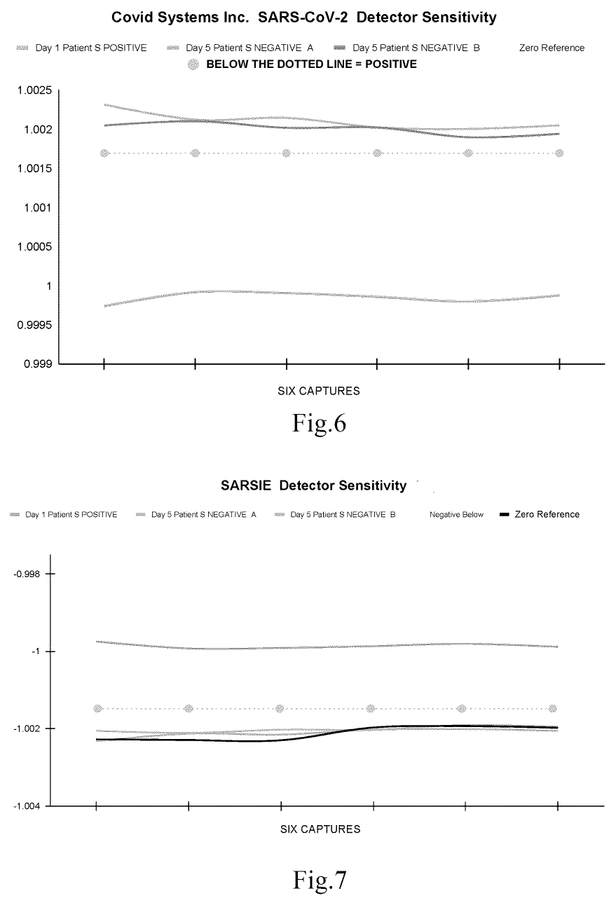

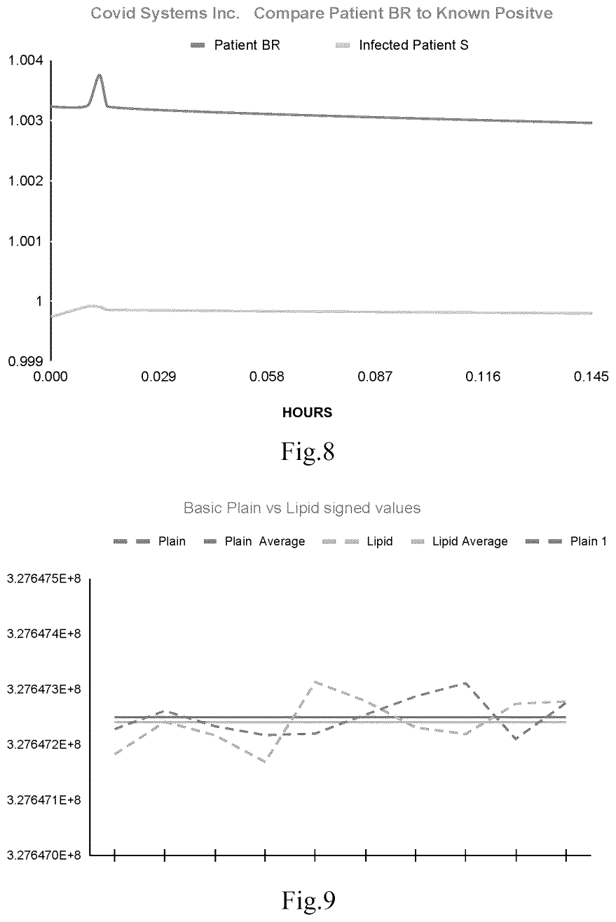

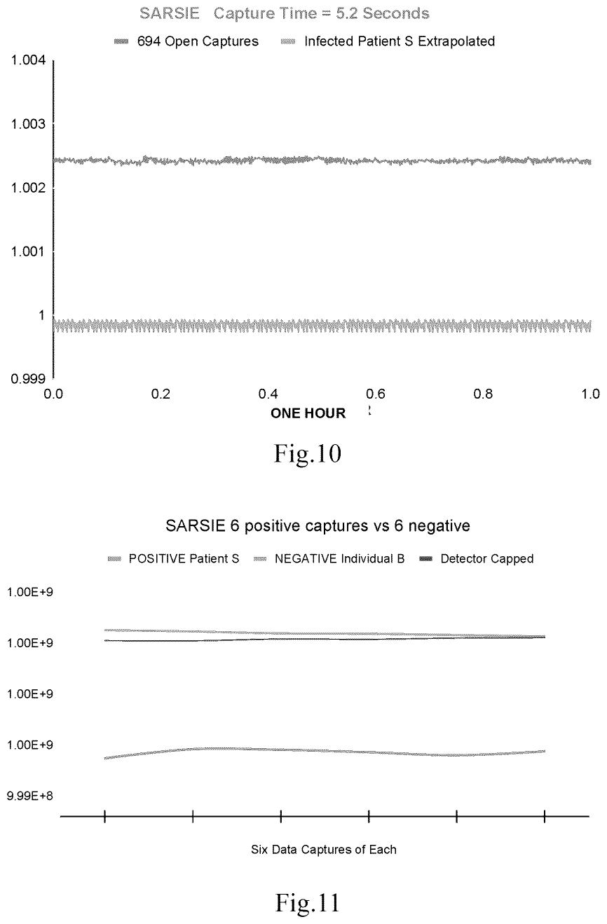

FIG. 4 graphs five 50 millisecond breath tests of four subjects and one “Room Tone” which is the floor value for this test. The “DELTA” value shows a clear separation between positive and negative.

In various embodiments, the devices described herein can be used in a lone or smartphone mode. The data capture for each reading is about 20K 16-bit values. The embedded processor's micro SD memory supports at least 500 readings which can be increased as needed. The Sarsie code is written in C (standalone) and Swift (iPhone).

Each Sarsie's output is the raw data and also a single value which is the integrated sum of all the reading values, the virus load for this reading. These are stored internally (see “readings” above) and transmitted as needed on Sarsie's wi-fi port along with subject ID, GMT time, GPS geolocation, and other information as required.

Technology: In various embodiments a IEEE 802.1 wireless built in can be used, a Micro SB memory such that at least five hundred SARSIE readings can be used in some embodiments.

In operation, the device's red LED light source 14 on PC Board 3 shines crosswise to the subject's breath flowing through the breath passage. Sensor photodiodes 12 and 13 on PC Board 3 measure the intensity of the light reflected as described above. Analysis to determine whether the subject is negative or positive is computed by the CPU on PC Board 3. The test result is displayed by the indicator LED 5 and saved to non-volatile memory on PC Board 3.

Data and results can be sent immediately via an electronic transmitting means, such as Bluetooth 8, as noted above to a linked smartphone app or cloud storage to assist in automated contact tracing.

It will be clear to those skilled in the art that embodiments such as device shape and construction, number and arrangement of sensors, light source LED details, purge fan details, direct radio link in addition to Bluetooth, and other such embodiments can be adjusted if needed for any of several applications within the scope of this invention.

In various embodiments, described herein is an These drawings show the amount of light reflected from airborne virus microdroplets as they pass through the invention's central bore. This value is the viral load at the moment of testing. It is also possible, for purposes of comparison, to simulate positive infection with an oral rinse of 1000:1 water to a surfactant. This appears as “surfactant” in the drawing descriptions. Live human subjects are identified with initials. FIG. 12 shows the detailed operation and breath sampling of the Sarsie hardware.

In these graphs, the Y axis values always represent the same kind of value, viral load, but for reasons of clarity and comparison the presentation may be scaled or the value inverted. This is shown on each graph. Accuracy of the Sarsie sensors is at least 16 bits.

### Applications Testing Water:

In various embodiments, the devices and methods can be adapted for use testing water for viruses can be detected, as described herein in various embodiments.

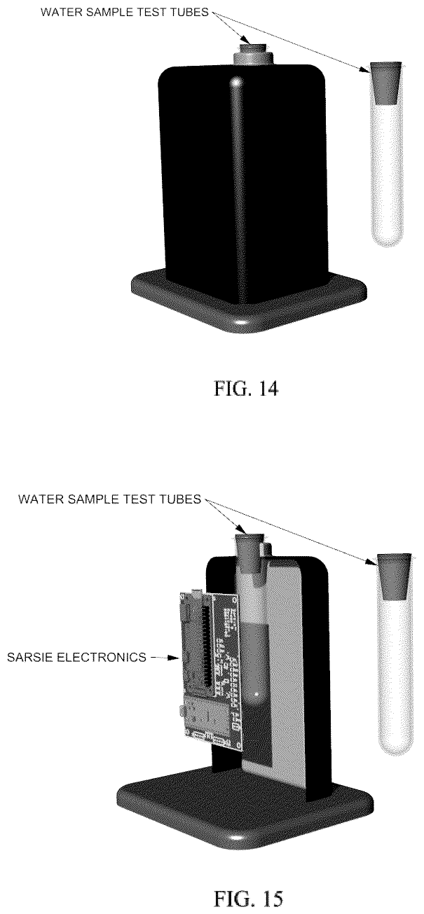

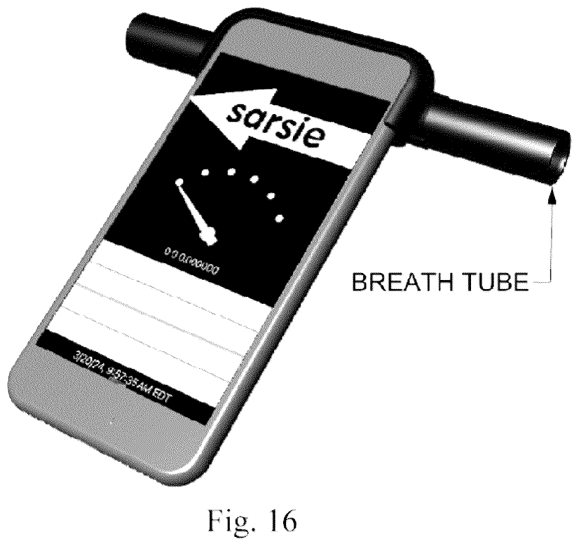

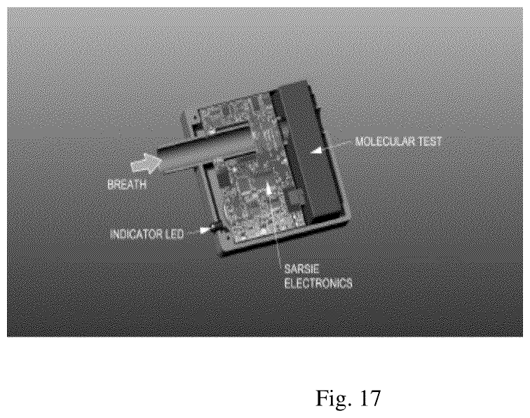

FIG. 14 shows the device as adapted for water applications in sealed enclosure. A water sample is presented for testing in a glass test tube. FIG. 15 shows in detail how the test tube is slipped into the device's central bore. A circuit board, as in other implementations described herein, is the same as in the other Sarsie devices except for being turned 90 degrees facing upward to receive the water-filled test tube. This is the arrangement for single samples. For running water such as in a sink faucet, the test tube is removed which clears the device's central bore for a faucet nozzle or hose as appropriate.

It should be understood that the Sarsie device here is the same unit as for its other uses having the same sealed enclosure and through bore. This works because the atmosphere and clear water are similar optically. The viruses of interest all extrude lipid envelopes which are by their nature waterproof.

In some preferred embodiments, the invention is a battery-powered sealed device about the size of a computer mouse. It has an actuator button and a multicolor indicator LED (light-emitting diode). In operation, the said indicator LED lights up to show READY (repeating white strobe), TESTING (flashing blue), COVID NEGATIVE (green), and COVID POSITIVE (flashing red). A disposable tube is provided to reduce personal contact and the risk of transmitting the virus. If a subject tests positive, the device's program switches the device off or otherwise disables it to prevent re-use until it is sterilized and reset. A USB port is provided for charging batteries and programming.

To reduce the risk of infecting subsequent subjects, a purging fan and filter are provided to cleanse the breath passage and create negative pressure inside the device to capture stray virus particles. A filter can also be used, in some embodiments, to capture virus and enable molecular identification of viruses.

To run a test, the subject simply taps the actuator button and exhales for two seconds into the device's breath passage or into a disposable tube. The indicator LED flashes blue as the test result is computed. In four seconds, it turns either GREEN for negative or FLASHING RED showing the subject is infected. In addition to this immediate visual indication, the results can be sent simultaneously via the device's built-in Bluetooth to, for example, a linked smartphone app, along with other data which may be useful such as GPS geolocation for contact tracing.

### Smartphone

In varies embodiments provided herein, the device can be used with a smartphone. In the smartphone embodiment described herein, the necessary hardware is in the smartphone. For Smartphone applications, in some embodiments, the camera's flash can act as light source and the phone's camera as sensor. The sample space is in the 3D printed clip that is, in various embodiments, attached. Its enclosed bore is the same as other embodiments of Sarsie, as described herein, in various embodiments. Because the software can be used to read all the pixels of a smartphone image, the accuracy using smartphone applications is very high.

In various embodiment, the Sarsie app has been implemented for Apple and Nokia smartphones, giving the smartphone user Sarsie virus detection capability for pennies, the cost of an injection molded light clip. Smartphones' hardware and processing support the Sarsie app very well. One considerable advantage of this app embodiment of Sarsie is that a user has instant virus detection at hand and at all times without carrying a separate device. The apps' indicator needle instantly shows the quantity of viruses detected in the breath and at the same time keeps a moving scroll of history, so it is it easy to spot trends as viral shedding fluctuates. FIG. 16 shows a smartphone embodiment of Sarsie.

SARS-COV-2 is one of a group of viruses which reproduce by extruding a lipid (soaplike) bubble containing new virus particles. These tiny bubbles reflect light proportional to their concentration in the subject's breath. Measuring this reflected light to detect, and in various embodiments quantify, virus is central to the inventors' contribution described herein in various embodiments of the invention.

In its simplest form, the invention has one light source and an optical sensor. In various embodiments, features such as light sources, sensors, and processors to, for example, sense UV detect other pathogens, and increase processing speed can be incorporated.

In various embodiments, the invention provides for detecting enveloped viruses, also known as membrane bound viruses, by reflected light. When an enveloped virus particle, such as a COVID particle, becomes airborne, it forms a protective envelope of a lipid surfactant similar to soap. Like soap bubbles, this protective envelope reflects light. The brightness of this reflection is proportional to the reflecting surface area arising from virus concentration in a subject's breath.

When a COVID particle becomes airborne, it forms a protective envelope of a lipid surfactant similar to soap. Like soap bubbles, this protective envelope reflects light. The brightness of this reflection is proportional to the reflecting surface area arising from the virus concentration in the subject's breath.

The invention, in various embodiments described herein, is applicable to the class of viruses known as enveloped viruses. Examples of enveloped viruses include coronaviruses (CoVs). In various embodiments described herein, airborne viruses that are enveloped virus can be detected. Airborne respiratory viruses that infect humans, or other animals, that can be detected using Sarsie, as in various devices, apparatus, and methods described herein in various embodiments, include, for example, SARS-COV-2 and its variants, Influenzas A and B, Severe Acute Respiratory Syndrome (SARS), Middle East Respiratory Syndrome (MERS), Respiratory Syncytial Virus (RSV), H5N1 Avian Influenza, and variants thereof. Application of the approach described herein in various embodiments to the H5N1 “bird flu” is particularly valuable because this virulent virus has been increasingly shown to cross the species barrier from birds to mammals, including mink, ferrets, cattle, and now humans. The invention described herein in various embodiments is applicable to testing humans and other animals, including cattle, pigs, sheep, goats, and chicken. The devices, methods, and apparatus described herein are applicable to detecting airborne enveloped viruses wherever they are found.

In some embodiments, the Sarsie, and various embodiments described herein, can be used for animals, such as farm animals. Sarsie, for example, in the context of cows, could, in some embodiments be referred to as COWSIE, or SARSIE for cows. Cows have been in the current news because of the recent reports of the spillover of avian influenza H5N1 virus to dairy cattle. Avian influenza H5N1 is a highly pathogenic virus and has resulted in the death of meillions of domestic birds and thousands of wild birds in the United States. See Caserta, L. C., Frye, E. A., Butt, S. L. et al. Spillover of highly pathogenic avian influenza H5N1 virus to dairy cattle. Nature (2024), published Jul. 25, 2024.

### Implementation for Cows:

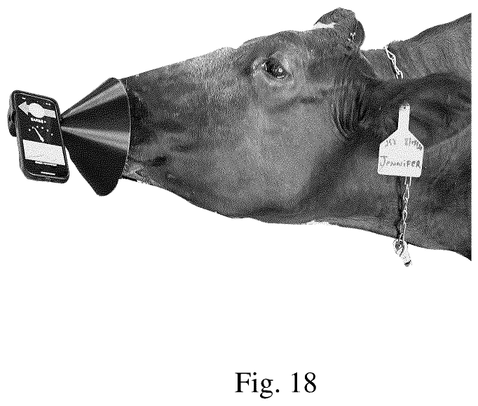

While the methods and devices described herein in various embodiments, are applicable to most animals, FIG. 18 is an illustration with a cow.

In FIG. 18, a SARSIE device is shown with a cow. For a SARSIE device with a cow, or COWSIE, additional elements are, in various embodiments, are used for the implementation of this method and device.

COWSIE demonstrates the flexibility of the SARSIE device concept to different applications. For a cow application, an enlarged conical nosepiece, or cone, to fit the cow's nose rather than using a breath tube as in the human context . . . instead of Sarsie's breath tube. An ultrasound proximity sensor such as the ADAFRUIT 3942 is mounted inside the cone. A reading is triggered when a cow's nose is approximately 2 inches in. Facial recognition technology can be included to identify the cow if it is not chip-enabled.

In addition, it has been observed that cows are very curious and will all sniff the COWSIE device without prompting. In various embodiments, the device can be left in the field to gather data unattended so the cows do the work of collecting samples as they each approach the device and put their noses into the nosepiece. Single readings can be triggered with the device's button.

In is understood by persons of skill in the art that the way this device is shown implemented for cows can be adapted to other farm animals, such as chickens, goats, sheep, and pigs.

In various embodiments, the devices described herein can be tailored to comprise molecular tests, or diagnostic methods, to identify the virus detected. These embodiments provide for rapid detection of viruses using Sarsie, or other embodiments described herein, followed by using diagnostic methods known to persons of skill in the art such as reverse transcriptase polymerase chain reaction (RT-PCR), antigen detection, nucleic acid amplification tests, and serology tests. These molecular tests can be incorporated directly into the devices, as described herein, or be implemented separately.

In various embodiments, provided herein is a battery-powered sealed device a little bigger than a computer mouse and weighing about ten ounces. The device is equipped with wireless communication means and an indicator LED. In various embodiments, the subject breathes into the device's axial bore. First, breath is scanned by optical sensors for reflected light. In some embodiments, breath temperature data is collected. This is step 1. In step 2, further along the axial bore, the breath enters the molecular test. Depending on the choice of molecular test, this could be, for example, direct chemical interaction of the breath with a sensor inside the device. In some embodiments, four phases of the device, provided in various embodiments, can be described as follows: Phase I is an Idle phase, the device is switched off with essentially zero battery drain while waiting. To start a test, tap the device gently on a hard surface, flick it with a finger, or press its optional button. Recording begins immediately. In Phase II, Recording, several seconds of breath data are captured including temperature. During recording the LED pulses blue. The subject needs no particular instruction about breathing; the test works equally well with gentle or fast breath. Exhaled breath in humans is fairly stable around 93.2 degrees Fahrenheit. This is accurately measured, in some embodiments, by the device's temperature sensor. If it is out of range, the capture data is rejected. In effect, breath temperature acts as quality control of the data. In particular, a subject cannot fake a negative reading by not breathing. In Phase III, measurement of light reflection, after analysis of the captured data the LED lights up GREEN for negative or RED for positive. If the result is negative, virus free, the LED stays

GREEN for ten seconds and then reverts to idle. The molecular test is not necessary and the device is ready to re-use. The subject is free to go. But if the subject tests positive, infected, the LED holds on RED as the molecular test starts automatically.

In various embodiments, a 2-step test can be used to detect and then characterize the envelope virus. In step 1, data are captured and analyzed to determine whether a subject is positive for an envelope virus or negative. In various embodiments of Sarsi, a green light indicates negative, virus fee, and a red light indicates a positive test for virus. In some embodiments, where the subject is negative, virus free, the LED reading stays green for ten seconds and reverts to idle and the subject is free to go, but if the subject tests positive, indicating an infection, the LED holds on red as molecular test starts automatically. The molecular test can be any method known in the art including polymerase chain reaction (PCR), or other nucleic acid amplification tests, reverse transcriptase polymerase chain reaction (RT-PCR), antigen detection, a serology test, or combinations thereof. Depending on the type of the molecular test, in some embodiments, the device's on-board processor can evaluate the subject's breath directly and report the result in as little as an hour. On completion, measures appropriate for an infected subject can be taken such as reporting geolocation via wireless communication for contact tracing and/or routing the infected subject into quarantine, and disinfecting the test device.

What is claimed is:

1. A device for detecting an enveloped virus in a subject, comprising:
a sample space through which breath from the subject passes;
a light source positioned to shine through the sample space, and
one or more sensors positioned to measure the light from the light source which is reflected from the virus in the sample space.

2. The device of claim 1, wherein the sample space comprises a transparent tube.

3. The device of claim 2, wherein the transparent tube is a disposable tube.

4. The device of claim 1, wherein the subject is a human.

5. The device of claim 1, wherein the subject is a cow.

6. The device of claim 1, wherein the one or more sensors are digital optical sensors.

7. The device of claim 1, wherein the one or more sensors optical sensors have an accuracy of around one part per 65536 and 16 bit integrated circuits.

8. The device of claim 1, wherein the light source a LED (light emitting diode).

9. The device of claim 1, further comprising a purging fan.

10. The device of claim 1, further comprising a filter to capture virus particles.

11. The device of claim 1, further comprising a molecular test to identify the enveloped virus detected.

12. The device of claim 11, wherein the molecule test is selected from reverse transcriptase polymerase chain reaction (RT-PCR), antigen detection, nucleic acid amplification tests, and serology tests, or combinations thereof.

13. The device of claim 1, further comprising a wireless means to communicate externally.

14. The device of claim 1, wherein the device comprises a smartphone.

15. A method of detecting an enveloped virus in a subject, comprising:
having the subject breath into a sample space;
shining light on the sample space;
measuring the reflected light in the sample space to determine the presence of the enveloped virus.

16. The method of claim 15, further comprising quantifying the enveloped virus.

17. The method of claim 15, further comprising identifying the enveloped virus using a molecular test.

18. The method of claim 17, wherein the molecular test is selected from reverse transcriptase polymerase chain reaction (RT-PCR), antigen detection, nucleic acid amplification tests, and serology tests, or combinations thereof.

19. The method of claim 15, wherein the enveloped virus is selected from the group consisting of SARS-COV-2, Influenzas A and B, Severe Acute Respiratory Syndrome (SARS), Middle East Respiratory Syndrome (MERS), Respiratory Syncytial Virus (RSV), H5N1 Avian Influenza, variants thereof, and combinations thereof.

20. The method of claim 15, wherein the light is in the range of 380 to 780 nm.

21. The method of claim 15, wherein the sample space comprises a transparent tube.

22. The method of claim 15, wherein the subject is a human.

23. The method of claim 15, wherein the subject is a cow.

24. A device for testing water for enveloped viruses, comprising a sample space for presenting a water samples;
a light source positioned to shine through the sample space, and
one or more sensors positioned to measure the light from the light source which is reflected from the virus in the sample space.

25. The device of claim 24, wherein the sample space is a central bore adapted for receiving running water.

26. The device of claim 24, wherein the sample space further comprises a transparent test tube for providing a water sample.

27. The device of claim 26, wherein the transparent test tube is glass.
